(19) United States
(12) Patent Application Publication
(10) Pub. No.: US 2007/0292860 A1
(43) Pub. Date: Dec. 20, 2007

(54) Staphylococcus Aureus Specific Diagnostics

(76) Inventors: Frank Henri Johan Schuren, Veenendaal (NL); Jan Verhoef, Zeist (NL); Roy Christiaan Montijn, Amsterdam (NL)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/587,884

(22) PCT Filed: Apr. 29, 2005
(86) PCT No.: PCT/NL05/00326

§ 371 Date: Jul. 16, 2007

(30) Foreign Application Priority Data
Apr. 29, 2004 (EP) 04076309.6
May 10, 2004 (EP) 04076394.8

(51) Int. Cl. C12Q 1/68

(52) U.S. Cl. 435/6

(57) ABSTRACT

A method for typing sample nucleic acid derived or obtained from a Staphylococcus aureus strain, comprising providing an array comprising a plurality of nucleic acid molecules, wherein said plurality of nucleic acid molecules is derived from a first set of at least two different strains of Staphylococcus aureus, providing at least two different reference hybridization patterns by hybridizing said array with at least two different reference nucleic acids obtained or derived from a second set of at least two different strains of Staphylococcus aureus, wherein said strains of Staphylococcus aureus in said second set are separable into at least two groups on the basis of a value for at least one phenotypic parameter, creating at least two different clusters of reference hybridization patterns by clustering the reference hybridization patterns by unsupervised multivariate analysis, hybridizing the same array as used for preparing the reference hybridization patterns with sample nucleic acid to obtain a sample hybridization pattern, and assigning the sample hybridization pattern to one of said at least two different clusters of reference hybridization patterns.

### TECHNICAL FIELD

The invention relates to the fields of diagnostics for Staphylococcus aureus, more in particular to array-based methods of typing Staphylococcus aureus strains.

### BACKGROUND OF THE INVENTION

Staphylococcus aureus is a major problem in the care of hospitalized patients. There are many different Staphylococcus aureus strains, some of which are resistant to a wide spectrum of antibiotics. The different strains of Staphylococcus aureus behave differently with respect to their infectiousness. Some strains rapidly spread from patient to patient, whereas other strains do not spread that easily. Considering that at least some of the Staphylococcus aureus strains are capable of causing severe disease in hospitalized patients, hospitals maintain very strict hygiene rules. It has previously not been possible to quickly determine whether a particular strain is a fast spreading strain (epidemic strain) or not. A hospital that is faced with a methicillin resistant Staphylococcus aureus (MRSA) infected patient thus has no choice but to enforce the most stringent quarantine and hygienic rules.

Array technology has become an important tool in various fields related to biology and medicine. Several types of arrays have been developed through the years. With the advent of miniaturization and automation more and more information has been entered into arrays. The current trend in array technology is to generate ever-larger arrays, carrying more and more information on them.

In array-based diagnostics, the hybridization pattern, or the pattern of intensities with which the various spots on the array hybridize to the sample nucleic acid, contains the data which is to be compared to that of another sample nucleic acid. In conventional arrays, the number of nucleotides per spot is preferably kept as low as possible for reasons of economy and precision.

A higher level of information contained on an array is used primarily to provide for more detailed analysis of nucleic acid samples, i.e. to make visible or reveal the minutest differences between two such samples that are to be compared. For instance, in human diagnostics, arrays are used to classify groups of patient having the same disease, but having different prognosis, and thereupon reveal the genes that are responsible for this difference in prognosis. Such experiments are mostly performed on the basis of expression arrays, because only the level of expression of a certain gene is believed to provide for the necessary resolution to distinguish between the two groups of patient, i.e. to provide for sufficient discriminatory power between them.

In these diagnostic methods, known as expression profiling, the nucleic acid used to probe the array—i.e. the expressed mRNA—provides for complex nucleic acid. The introduction of larger numbers of nucleotides in the array now introduces another difficulty, particularly when complex nucleic acid is used to probe the array. In the situation of expression profiling, a large number of spots have signals between the value 0 and 1, indicative for the fact that not all of the nucleic acid in the spot is hybridized to probe nucleic acid, which is a feature used to determine or quantify the level of expression of the genes involved.

Eventually, in comparing the different hybridization patterns, decisions have to be made which signals of the array are included in the analysis and which are left out. Usually this occurs on the basis of cut-off values, introduced to bias the analysis towards inclusion of the most notable or largest changes in intensity of certain spots. A pivotal role in this process plays the reference pattern, the pattern to which the pattern generated with the test material or sample nucleic acid is compared. A problem with the methods of the prior art is now that expression of nucleic acids represents a state the organism is in, which means that the same organism can have different expression patterns depending on the circumstances. The prior art methods are thus less suited to provide for typing of organisms irrespective of their metabolic state.

### SUMMARY OF THE INVENTION

The present inventors have now found a method of preparing reference hybridization patterns that provides for such a high discriminatory power that it allows for a level of typing of sample nucleic acids that is surprisingly detailed. For instance, the present inventors have now devised a method of typing that allows for sample nucleic acids of different bacterial strains to be typed at the level of such detailed phenotypic parameters as epidemicity, whereas the typing itself occurs on the basis of whole-genome-array differential hybridization. In such whole-genome-array differential hybridization approaches, both the nucleic acid molecules on the array and the sample nucleic acid consist of (random) genomic DNA fragments. That this level of detail is attained is surprising now that one would not expect that distinguishing between epidemic and non-epidemic subtypes among strains of Staphylococcus aureus would be possible based on the composition of the genomic DNA.

In one aspect the present invention provides means and methods with which it is possible to quickly determine whether an MRSA strain is epidemic or not. The invention is also suited to determine other characteristics of particular Staphylococcus aureus strains. To this end the invention provides specific arrays that are capable of distinguishing between the various strains of Staphylococcus aureus and more importantly, to estimate properties of a particular Staphylococcus aureus strain, even under circumstances wherein hybridization patterns generated on the array are not identical to an already earlier generated hybridization pattern.

Thus in one aspect the invention provides a method method for typing sample nucleic acid derived or obtained from a Staphylococcus aureus strain, comprising:

providing an array comprising a plurality of nucleic acid molecules, wherein said plurality of nucleic acid molecules is derived from a first set of at least two different strains of Staphylococcus aureus;

providing at least two different reference hybridization patterns by hybridizing said array with at least two different reference nucleic acids obtained or derived from a second set of at least two different strains of Staphylococcus aureus, wherein said strains of Staphylococcus aureus in said second set are separable into at least two groups on the basis of a value for at least one phenotypic parameter;

creating at least two different clusters of reference hybridization patterns by clustering the reference hybridization patterns by unsupervised multivariate analysis;

hybridizing the same array as used for preparing the reference hybridization patterns with sample nucleic acid to obtain a sample hybridization pattern, and

assigning the sample hybridization pattern to one of said at least two different clusters of reference hybridization patterns.

In one preferred embodiment the average size of the fragments in said sample nucleic acid is between about 50 to 5000 nucleotides.

In another preferred embodiment the average size of the molecules in said plurality of nucleic acid molecules is between about 200 to 5000 nucleotides.

In still another preferred embodiment the array comprises between about 1.500 and 5.000 nucleic acid molecules randomly chosen from fragments of the fragmented genomic DNA of said at least two different strains of Staphylococcus aureus.

In still another preferred embodiment said sample nucleic acid is derived from a pure culture of Staphylococcus aureus.

In still another preferred embodiment said plurality of nucleic acid molecules is derived from at least three, preferably at least 5, and even more preferably at least 8 different strains of Staphylococcus aureus.

In still another preferred embodiment, the method comprises comparing the sample hybridization pattern with at least 3, more preferably at least 5 and even more preferably at least 50 different reference hybridization patterns.

In still another preferred embodiment, the phenotypic parameter is the epidemicity of said Staphylococcus aureus strains.

In still another preferred embodiment, the comparison comprises Partial Least Square-Discriminant Analysis (PLS-DA) of the reference hybridization patterns together with the sample hybridization pattern and wherein at least one phenotypic parameter of which the values are known for the reference hybridization patterns, (and which information is used to supervise the PLS-DA analysis), is additionally determined or estimated for the sample nucleic acid or the source it is derived from.

In still another preferred embodiment, the method further comprises clustering patterns based on the supervised PLS-DA analysis.

In still another preferred embodiment, the method further comprises typing said sample nucleic acid on the basis of the presence or absence in a cluster.

In still another preferred embodiment, essentially all reference hybridization patterns are generated by nucleic acid of Staphylococcus aureus strains.

In still another preferred embodiment, the typing comprises determining whether the Staphylococcus aureus in said sample nucleic acid is derived from is an epidemic strain.

In another aspect, the present invention provides a kit of parts, said kit comprising a combination of an array as described herein above, and wherein said kit further comprises at least two different reference hybridization patterns or reference nucleic acids derived from strains of Staphylococcus aureus as described herein above.

### BRIEF DESCRIPTION OF THE DRAWINGS

FIG. 1 presents a list of S. aureus strains and their epidemicity characteristics. Each MRSA strain is identified by a unique TNO Type Collection number (TTC nr, 1st column). Each strain was characterized as S. aureus strains by Riboprint classification (2nd column). Epidemic character was determined from daily hospital practice (3rd column).

FIG. 2 shows the clustering for epidemicity of MRSA strains by supervised PLS-DA analysis of whole-genome-array differential hybridization data. Cy-labeled genomic DNA of 19 different MRSA strains was hybridized to arrays containing a representation of the S. aureus genome. The quantified fluorescent hybridization patterns of the S. aureus strains, representing a highly complex n-dimensional data-set, were analyzed with Partial-Least-Square-Discriminant-Analysis (PLS-DA) on basis of the known epidemic character of each MRSA strain. The PLS-DA plot below shows single point projections of each single-strain complex hybridization pattern in a 2-dimensional plane (small circles, with text indicating strain TTC.03 number mentioned in FIG. 1). In duplo hybridized strains are indicated with bold text. Based on a specific part of the dataset, the PLS-DA analysis is able to cluster the strains in two separate clusters (manually placed ellipses, indicated E=Epidemic and N=non-epidemic) according to their known epidemicity. Note: Cy5/Cy3-ratio's were transformed to a 0 and 1 dataset by cut-off 0.5. PLS-DA-scaling was by mean centering. The non-epidemic strain “236” was positioned in-between the E- and N-cluster by PLS-DA. Numbers of datasets refer to the strain numbers shown in FIG. 1.

FIG. 3 highlights aspects of Model complexity as described hereinbelow in the section on Partial least squares (PLS) analysis methods.

FIG. 4 illustrates aspects of discriminant analysis as described hereinbelow in the section on Principal Component-Discriminant Analysis (PC-DA). In this figure, D is the discriminant axis, P is a projection line, X₁ and X₂ are two variables and x and o represent samples from two different groups.

### DETAILED DESCRIPTION OF THE INVENTION

It is a feature of the present invention that together with (e.g. simultaneously, prior to or after) the genetic comparison between a sample nucleic acid and reference nucleic acid, at least one phenotypic parameter (e.g. the epidemicity) is determined for each source of reference nucleic acid, which phenotypic parameter is then used in the statistical classification of the data.

The present invention uses differential hybridization to classify sample nucleic acids in general and uses in particular whole-genome differential hybridization to classify organisms. The present invention in one embodiment relates to a method employing an array of random genomic DNA fragments from a pool of different strains of Staphylococcus aureus to classify “new” strains of Staphylococcus aureus according to clinically relevant features (such as antibiotic resistance, epidemicity, virulence, pathogenicity, etc.).

Thus, where the present invention prescribes that at least two different strains of Staphylococcus aureus must be separable into at least two groups on the basis of a value for at least one phenotypic parameter of interest, a method of the invention will in a preferred embodiment allow for the distinction between epidemic and non-epidemic subtypes.

The additional step of providing information on at least one phenotypic feature for the reference strains of Staphylococcus aureus provides in one aspect of the invention a method that uses an a-specific collection of e.g. genomic DNA fragments from a group consisting of different strains of Staphylococcus aureus to cluster according to similarity and classify the hybridization pattern obtained with e.g. the gDNA of unknown members within or outside said group, and that is capable of further distinguishing or separating those clusters based on at least one phenotypic feature.

The term a-specific is used deliberately because the array of the present invention provides an analysis tool that is not necessarily suitable only for the analysis of nucleic acids of strains of Staphylococcus aureus that are related that of the different strains of Staphylococcus aureus spotted on the array, but provides in principle sufficient discriminatory power to allow for the analysis of genomes that are taxonomically removed from or unrelated to the nucleic acids on the array. Yet, the best results and highest discriminatory power is achieved when selecting the nucleic acids for the plurality of nucleic acid molecules of the array such that the sample nucleic acid is highly related (i.e. that its hybridization pattern clusters in between or with the reference patterns).

The plurality of nucleic acid molecules on the array is derived from at least two different strains of Staphylococcus aureus, preferably the plurality of nucleic acid molecules is derived from at least three, more preferably at least 5, and even more preferably at least 8 different strains of Staphylococcus aureus.

An array nucleic acid molecule is typically a (usually single stranded) genomic DNA fragment of a strain of Staphylococcus aureus.

In a method of the present invention, different strains of Staphylococcus aureus used for the preparation of reference hybridization patterns are separable into at least two groups on the basis phenotypic characteristics or parameters, also termed herein a value for at least one phenotypic parameter of interest. The term “value” includes both quantitative and qualitative values. Thus, for instance, an array comprises genomic DNA fragments from an epidemic strain of Staphylococcus aureus as well as genomic DNA fragments from a non-epidemic strain. It has been found that this method is ultimately suitably for the rapid and accurate typing of different strains of Staphylococcus aureus. For instance, in the case of methicillin-resistant staphylococcus aureus (MRSA) it is even possible to distinguish epidemic from non-epidemic strains.

In a particularly preferred aspect of the invention therefore, nucleic acid obtained or derived from at least two different strains of Staphylococcus aureus is used to generate a reference hybridization pattern. Preferably, at least 5 and more preferably at least 50 reference hybridization patterns are generated by nucleic acid obtained or derived from different Staphylococcus aureus strains. In a particularly preferred embodiment essentially all reference hybridization patterns are generated by nucleic acid of Staphylococcus aureus strains.

This particularly preferred embodiment is preferably combined with a statistical analysis for comparing the reference and sample hybridization patterns. In this way it is possible to determine the chance that a strain of Staphylococcus aureus comprises a certain phenotypic characteristic or genotypic relatedness of some but not all strains of Staphylococcus aureus. The nucleic acid can be derived from RNA but is preferably derived or obtained from DNA. i.e. derived from the genome. Thus in a preferred embodiment of the invention nucleic acid molecules are derived from DNA.

The reference hybridization patterns are typically derived by hybridizing an array of the invention with reference organisms, wherein typically one strain of Staphylococcus aureus gives rise to one reference hybridization pattern. Of course, in order to determine the relationship between strains, said at least two different reference nucleic acids and sample nucleic acid are derived from different strains of Staphylococcus aureus. In one embodiment the present invention relates to a method of classifying a strain of Staphylococcus aureus comprising hybridizing the DNA of a test-strain of Staphylococcus aureus to a DNA-array of the invention comprising a large number of randomly chosen genomic-DNA fragments, which genomic-DNA fragments are derived from a mixture of at least two, preferably at least 3, more preferably at least 4, still more preferably at least 8 different strains of Staphylococcus aureus in order to classify the genomic DNA of said a test-organism amongst said reference organisms.

In a preferred embodiment the DNA-array comprises about 1.000 to about 10.000, preferably about 1.500 to about 5.000, most preferably about 1.800 to about 2.400, still more preferably about 1.900 to about 2.200 randomly chosen genomic-DNA fragments.

In a preferred embodiment the randomly chosen genomic-DNA fragments have a length of about 500 to about 5.000, more preferably about 1.000 to about 2.000, more preferably about 1.300 to about 1.800, more preferably about 1.400 to about 1.600 nucleotides. Thus, in a most preferred embodiment, a DNA array employed in a method of the present invention comprises about 3 megabases.

In another embodiment the invention relates to a method of classifying a microorganism. The method employs a DNA-array comprising a large number (about 1000 to about 10.000, preferably about 1.500 to about 5.000, most preferably about 1.800 to about 2.400, still more preferably about 1.900 to about 2.200) randomly chosen genomic-DNA fragments (preferably having a length of about 500 to about 5.000, more preferably about 1.000 to about 2.000, more preferably about 1.300 to about 1.800, more preferably about 1.400 to about 1.600 nucleotides), which genomic-DNA fragments are derived from a mixture of at least two, preferably at least 3, more preferably at least 4, for instance 5, 6 or 7, still more preferably at least 8 different micro-organisms in order to classify the genomic DNA of a microorganism. The mixture may suitably represent a gDNA pool of different strains of Staphylococcus aureus.

The method of the present invention preferably uses whole-genome arrays in order to investigate or determine the presence or absence of comparative (i.e. complementary) DNA regions in other strains of Staphylococcus aureus by hybridization. In contrast to the prior art methods, the present invention preferably does not employ so-called open-reading frame (ORF)-probes as nucleic acid molecules on the array. Such probes are derived from and only detect fragments of specific genes, or gDNA fragments of Staphylococcus aureus. Instead, the present invention preferably employs digested genomic DNA to obtain double-stranded gDNA fragments, which fragments are then preferably denatured to serve as single-stranded random gDNA probes that may be formed in a plurality of nucleic acid molecules suitable for construction of an array of the invention. In another preferred embodiment of a method of the present invention for typing Staphylococcus aureus DNA, a further improvement over the prior art methods is realized by providing an array of random genomic-DNA fragments derived from a gDNA pool of various and different strains of Staphylococcus aureus. This has the advantage that with a single experiment or assay the relationship can be established between the test organism and a group of reference organisms having a defined phenotypic characteristic.

The present invention now ultimately allows for the study of multigene features in Staphylococcus aureus. The herein described approach thus supports or allows for the incorporation of the classification of phenotypic characteristics of Staphylococcus aureus, such as for instance antibiotic resistance of the test-organism regardless of the genetic basis thereof. Thus, when applying the present method to the classification of Staphylococcus aureus and including at least one clinically relevant parameter for said Staphylococcus aureus strains (e.g. antibiotic resistance or epidemicity) it is not only the genotypic characteristics that classify the strains, but the combined genotypic and phenotypic characteristics of that strain, irrespective of any causative relation between the two.

It is not necessary to have detailed knowledge of the sequences that are present on the array. In the present invention patterns are compared with each other.

In order to construct an array containing a genome-wide representation of at least two different Staphylococcus aureus strains, a mixed-genomic library of at least two strains may be made by mixing gDNA of said at least two strains. Preferably, strains are selected that showed each a different value for a phenotypic parameter, e.g. a different profile of resistance to a broad set of antibiotics, preferably together covering most types of antibiotic-resistance. Preferably, the organisms do not contain significant plasmid bands in an agarose-gel analysis of their isolated gDNA. The gDNA mix may then be fragmented (e.g. sheared by sonication) and the fragments may be separated for instance in an agarose gel. DNA-fragments of appropriate sizes, preferably about 1-3 kb, may then be isolated (e.g. by excision from the gel and binding to a solid carrier such as glass-milk). A suitable number of gDNA fragments that is randomly retrieved from the gDNA mixture and thus number may range from about 1.000 to about 10.000, preferably about 1.500 to about 5.000, most preferably about 1.800 to about 2.400, still more preferably about 1.900 to about 2.200 randomly chosen genomic-DNA fragments. The effect of the gDNA mixture of multiple strains is that upon isolation of DNA fragments therefrom, a random pool of fragments from the various strains is obtained, which is used to construct the array.

The randomly chosen isolated fragments are preferably further multiplied to provide for a proper stock of material. Multiplication of the fragments may for instance be performed by a combination of cloning and nucleic acid amplification techniques as described in Example 1 below. The double stranded gDNA fragments may then be end-modified to allow their immobilization on the array surface, for instance by performing a PCR amplification reaction wherein one or both of the primers contain a free NH2-group coupled via a C6-linker to the 5′ end of the primer.

The randomly chosen, isolated and optionally amplified gDNA fragments may then be spotted on a surface to provide for a DNA micro-array. In order to facilitate coupling of the fragments, the surface of the array (e.g. the slide, the surface of which may i.a. be glass, gold, etc.) may be modified. Spotting may occur by any method available, for instance by using ElectroSpray Ionization (ESI) micro-array printing. After spotting of the fragments, the slide surfaces may be blocked to prevent further attachment of nucleic acids, e.g. by treatment with boro-anhydride in case of formaldehyde modified glass-slide surfaces.

Part of the original gDNA material of the individual strains is used to provide for material that can be hybridized with the array, i.e. to provide for reference nucleic acid. To facilitate detection of successful hybridization, the gDNA is suitably labelled, preferably fluorescently (e.g. by using Cy™ labels [Amersham Pharmacia Biotech]). Fluorescent labelling kits are commercially available from various manufacturers.

The average size of sample nucleic acid has an effect on the signal distribution on the array. Larger sample molecules comprise more information and are thus more likely to find a suitable hybridization partner in more of the spots. Reducing the average size of the sample nucleic acid can reduce this phenomenon. On the other hand, when the sample nucleic acid is too small, the nucleic acid fragments in the sample contain too little genetic information and also find suitable hybridization partners in many spots. The average size of the fragments in the sample nucleic acid is preferably between about 50 and 5000 nucleotides. More preferably, the average size of the fragments in the sample nucleic acid comprises a size of between about 50 and 1000 nucleotides, more preferably between about 50 and 500 nucleotides.

The sample nucleic acid preferably represents the whole sample genome. The hybridization pattern obtained with the sample nucleic acid on the array is compared with a reference hybridization pattern. The reference hybridization pattern can be artificially generated, for instance, through using knowledge of the nucleic acid composition of a reference sample, for instance the genome sequence of a Staphylococcus aureus strain of which the genomic sequence is known. However, in a preferred embodiment, the reference hybridization pattern is generated by hybridizing reference nucleic acid to the array. Comparison of the sample hybridization pattern with the reference can at least be used to determine whether the sample nucleic acid is the same or similar to the reference nucleic acid. This is useful when one needs to determine whether, for instance, the sample nucleic acid contains a particular Staphylococcus aureus strain. In this setting a reference hybridization pattern is generated with nucleic acid of the particular Staphylococcus aureus strain and when the sample hybridization pattern is essentially the same as the reference hybridization pattern, the sample is identified as containing the particular Staphylococcus aureus strain. In a preferred embodiment a method of the invention further comprises comparing the sample hybridization pattern with at least one other reference hybridization pattern. In this way the sample can be compared to at least two different reference nucleic acids. Of course, upon continued use of the array, more and more Staphylococcus aureus hybridization patterns are generated and all of these can be used to compare with the sample nucleic acid. Thus once a hybridization pattern is generated with sample nucleic acid this hybridization pattern can in a subsequent experiment be used as a reference hybridization pattern. Thus in a preferred embodiment a method of the invention further comprises comparing the hybridization pattern with at least 2, preferably at least 5 and more preferably at least 50 reference hybridization patterns. More preferably at least 100, more preferably at least 1000 reference hybridization patterns.

The sample hybridization pattern and the reference hybridization pattern can be a subset of signals obtained from the array. A hybridization pattern can consist of one signal; preferably the hybridization pattern consists of at least 20% of the signals obtained following hybridization to the array. More preferably, the hybridization pattern consists of at least 50% of the signals from the array. In a particularly preferred embodiment the hybridization pattern comprises at least 80% of the signals of the array.

A method of the invention cannot only be used to determine whether a Staphylococcus aureus sample nucleic acid is the same as a particular Staphylococcus aureus reference nucleic acid. The sample hybridization pattern can, as it happens, be different from any of the reference hybridization patterns. A particularly useful characteristic of the methods and arrays of the invention is that also in this case a method of the invention can provide useful information. Phenotypic characteristics associated with the Staphylococcus aureus strain from which the sample nucleic acid is derived or obtained are very often the result of the interplay of a large number of different sequences and/or genes. In these situations it is not possible to type a particular sample on the basis of the signal obtained in one or more spots. Rather, the signals of very many different spots need to be compared. The methods and arrays of the invention are particularly suited for this type of analysis. To this end the reference hybridization patterns and the sample hybridization pattern are analyzed using statistical software. In one embodiment, a method of the invention further comprises unsupervised multivariate analysis (e.g. Principal Component Analysis, PCA) of the Staphylococcus aureus reference hybridization patterns together with the hybridization pattern generated by the Staphylococcus aureus sample nucleic acid. Based on this analysis a hybridization pattern is given an n dimensional value (with n representing a value between 2 and the total number of datapoints included in the analysis), which can be reduced to its preferably 2 principal components. These components can be visualized in a multi-dimensional visualization, preferably in a two-dimensional visualization. The dimensional value of the components can be plotted for all hybridization patterns, which are included in the analysis whereupon the grouping or clustering of the preferably two-dimensional values of the hybridization patterns can be scrutinized. In a preferred embodiment the two-dimensional value of the sample hybridization pattern is compared with al two-dimensional values of the reference hybridization patterns. In this way it is possible to provide a statistical estimation of the relatedness of the Staphylococcus aureus strain that the sample nucleic acid is derived or obtained from compared tot the included references.

In a preferred embodiment the two-dimensional values of the reference hybridization patterns are clustered. This clustering is preferably done on the basis of the relatedness. This typing is typically associated with a margin of error for the classification, i.e. the chance that the sample nucleic acid is wrongly classified as being related to a particular (cluster) of reference strains. Thus a method of the invention preferably further comprises typing the relatedness of a Staphylococcus aureus used to generate said sample nucleic acid, on the basis of the presence or absence in a cluster.

The term “clustering” refers to the activity of collecting, assembling or uniting into a cluster or clusters items with the same or similar characteristics, a “cluster” referring to a group or number of the same or similar items gathered or occurring closely together. “Clustered” indicates that an item has been subjected to clustering. The process of clustering used in a method of the present invention may be done by hand or by eye or by any (mathematical) process known to compare items for similarity in characteristics, attributes, properties, qualities, effects, etc., through data from measurable parameters. Statistical analysis may be used.

Principal component analysis (PCA) can be performed with mean centering as the selected scaling method. Comparable results can be obtained using other scaling methods. In a preferred embodiment the mean centering scaling method is used. For a review of Principal component analysis reference is made to Joliffe IT. 1986. Principal Component Analysis. New York: Springer-Verlag.

It is a feature of the present invention that hybridization patterns are extended with further information on the Staphylococcus aureus strain wherefrom a reference and/or sample nucleic acid is obtained or derived. For instance, hybridization patterns may be extended with parameters that are determined in a way different from nucleic acid hybridization. For Staphylococcus aureus strains it is often important to know the antibiotic resistance phenotype of the strains. This resistance parameter can be added to the statistical analysis. The value of this parameter (resistant or sensitive, or further fine tuning) can be added to the hybridization pattern or to the statistical analysis of the hybridization patterns. The clustering can subsequently be based on this additional parameter. Thus in a preferred embodiment of the invention, at least one non nucleic acid based parameter is determined for the organisms that were the source of the reference hybridization patterns. The statistical analysis can subsequently be used to determine or estimate the value of this parameter for the Staphylococcus aureus that was the source of the sample nucleic acid. In this embodiment, a method of the invention further comprises Partial Least Square-Discriminant Analysis (PLS-DA) of the reference hybridization patterns together with the hybridization pattern generated by the sample nucleic acid, wherein at least one parameter of which the values are known for the reference hybridization patterns is used to supervise the PLS-DA analysis.

Partial Least Squares (PLS)

Partial least squares (PLS) has been described extensively in the literature (P. Geladi and B. R. Kowalski, Partial Least Squares Regression: A Tutorial, Analytica Chimica Acta, 185, 1986, 1-17. H. Martens and T. Naes, Multivariate Calibration, John Wiley & Sons, Chichester, 1989.) Whereas a principal component analysis (PCA) model has a descriptive nature, a PLS model has a predictive nature. In PLS, scores*loadings pairs, also called latent variables (LVs), are not calculated to maximise the explained variance in the predicting data set only, but also to maximise the covariance with the data to be predicted. The PLS model can be summarised mathematically by means of Equation (1) and Equation (2).
X=TP^{T}+E (1)
Y=TBQ^{T}+F (2)

Matrix X (also called X-block) represents a n*p matrix of independent variables (n chromatograms, for example, with p retention times per chromatogram), Y (also called Y-block) is a n*q matrix containing the dependent variables (concentrations, for example); P^{T} and Q^{T} are transpose S*p and S*q matrices, containing the dependent and independent variable loadings, respectively; T is an n*S matrix of S latent scores, B is a S*S matrix representing the regression of the scores of the X matrix on the scores of the Y-data; E and F are n*p and n*q matrices containing the residuals of the independent and dependent variables, respectively.

The standard error of validation (SEV) after extracting A LVs, is calculated from Equation (3).

$$SEV = \sqrt{\frac{{\sum }_{I = 1}^{{I}_{c}} {( {Y}_{i , j} - {y}_{i , j} )}^{2}}{{I}_{c}}} \qquad ( 3 )$$

where I_{c} is the number of calibration samples, y_{i,j} is the true value for the concentration of component j in object i; Y_{i,j} is the PLS predicted value for yᵢⱼ; q is the number of Y-variables. The extraction of LVs is continued as long as the SEV is improved significantly.

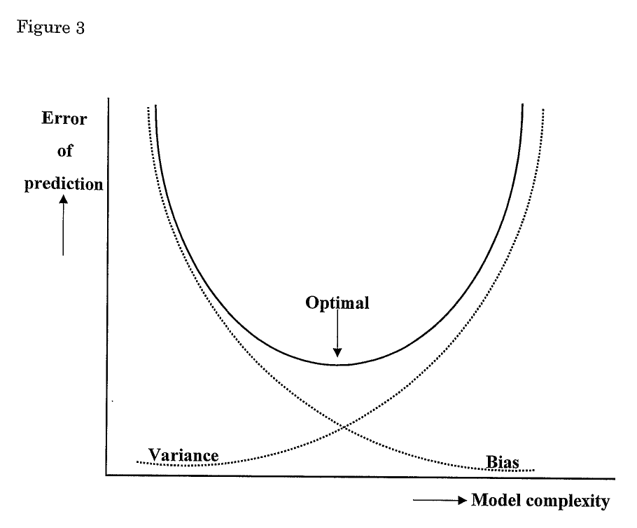

The number of LVs chosen must yield an optimal prediction of the variable of interest. However, there is a pay-off between variance and bias (or fit): a too complex model fits well, but may predict very poorly. This leads to the concept of optimal model complexity: an optimal balance between fit and variance is obtained. This is illustrated graphically in FIG. 3, where the increased complexity of the model is able to fit more features in the data, but the variance of the estimated parameters rises and the overall result attains a minimum value at the optimal model complexity.

Pure linear relations between X and Y will result in a simple model with usually two to five LVs. Complex non-linear relations can also be modelled. However, these will take up significantly more LVs to correlate Y to X.

Partial Least Squares—Discriminant Analysis (PLS-DA)

In PLS-DA classes (predefined groups) are used as dependent variables. The Y-block Y is a matrix of n*number of classes. The Y-block is filled with zeros and ones.

Example:

class=[1 2 2 1]

$$Y = [ \begin{matrix}1 & 0 \\ 0 & 1 \\ 0 & 1 \\ 1 & 0\end{matrix} ]$$

Using a Y-block in PLS which is filled with zeros and ones dependent on the class each sample belongs to, turns PLS into a discriminant analysis.

Principal Component-Discriminant Analysis (PC-DA)

Discriminant analysis (DA) [D. L. Massart, B. G. M. Vandeginste, L. M. C. Buydens, S. De Jong, P. J. Lewi and J. Smeyers-Verbeke, Handbook of Chemometrics and Qualimetrics: Part A, Elsevier, Amsterdam, 1997; B. G. M. Vandeginste, D. L. Massart, L. M. C. Buydens, S. De Jong, P. J. Lewi and J. Smeyers-Verbeke, Handbook of Chemometrics and Qualimetrics: Part B, Elsevier, Amsterdam, 1998] is applied if the interest is focused on differences between groups of samples. The technique is based on the assumption that samples of the same group are more similar compared to samples of other groups. The goal of DA is to find and identify structures in the original data, which show large differences in the group means. This process involves a priori knowledge of which samples are similar. Therefore, DA is said to be a supervised analysis technique. This distinguishes it from other unsupervised techniques such as principal component analysis (PCA), for example, which does not require a priori knowledge about samples.

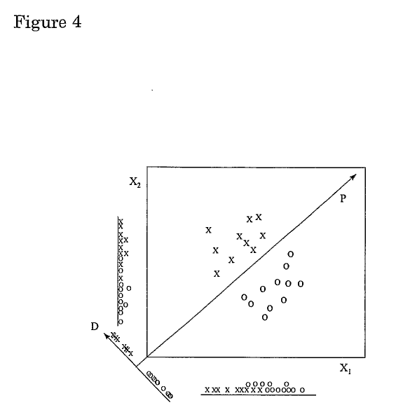

The first step in DA is to combine the original variables into a set of mutually independent new variables in such a way that the projection of the original samples in the space, spanned by a minimum number of these new variables, maximises the difference between the group means. This principle is demonstrated in FIG. 4. Two groups of samples are measured on two variables X₁ and X₂. Using the principal component (PC) maximum variance criterion these samples should be projected on the line through the samples as indicated in FIG. 4 by line P. For discriminating between the different clusters of samples this is not the optimal solution. However, the projection of the samples on line D shows a complete separation between the two clusters. The calculated factors are called discriminants or D-axes. All other projections give sub-optimal solutions. This is demonstrated in the FIG. 4 by comparing the projection of the samples on de D-line with those on the X₁ or X₂ axis.

DA describes most efficiently the differences between groups of samples. However, the number of variables is often large compared to the number of samples. This may lead to degenerate solutions. For instance three samples can always be separated by two variables independent of their similarity. If more samples are included this degeneracy effect will disappear. The general rule of thumb is that the number of samples should be at least four times the number of variables. This rule can lead to problems in the examination of nuclear magnetic resonance (NMR) spectra, for example. In the analysis of natural products the number of peaks (variables) per NMR spectrum is generally in the order of a few hundred. Under normal circumstances this would mean that one should measure at least 400 to 800 samples. In practice this never occurs. Based on this it would be impossible to perform DA on NMR spectra of natural products. However, there is a solution to this problem. Hoogerbrugge et al. [R. Hoogerbrugge, S. J. Willig and P. G. Kistemaker, Discriminant Analysis by Double Stage Principal Component Analysis, Analytical Chemistry, 55, 1983, 1710-1712.] developed a scheme in which the number of variables is reduced by PCA, firstly, followed by DA on the scores of the samples on the first PC axes. This technique is called principal component-discriminant analysis (PC-DA). Determining the exact number of PCs to include is difficult. The number should not be too small because including only the first few can result in a loss of a lot of the between-group information. The number should not be too large also, because it will exceed the number-of-samples-divided-by-four rule. Therefore, it seems advisable to include all PCs, which explain a significant amount of variance (for instance above 1% of the original variance) up to a maximum of the number of samples divided by four. If the total amount of variance explained by these PCs is very low then the number can always be increased. However, if the explained variance is low, the correlations between the original variables will be low also. As a consequence DA will generate a result which will be as complicated as the original problem.

The parameter used in the PLS-DA analysis is preferably a phenotypic parameter. The term “phenotypic parameter” is used here to define any parameter that describes any property of that is exhibited or expressed by the Staphylococcus aureus or a functional part thereof. Based on this analysis a hybridization pattern is given a n dimensional value (with n representing a value between 2 and the total number of discriminating datapoints included in the analysis), which can be reduced to its, preferably two, principal components for optimal correlation with the phenotypic parameter in a, preferably, two dimensional visualization. This preferably two-dimensional value can be plotted for all hybridization patterns whereupon the grouping or clustering of the preferably two-dimensional values of the hybridization patterns can be scrutinized. In a preferred embodiment the two-dimensional value of the sample hybridization pattern is compared with all two-dimensional values of the reference hybridization patterns. In this way it is possible to provide a statistical estimation of the chance that the Staphylococcus aureus strain that the sample nucleic acid is obtained or derived from comprises a certain phenotypic characteristic or not. This of course necessitates that this phenotypic characteristic is known for the Staphylococcus aureus strains from which the reference nucleic acid is obtained or derived. In a preferred embodiment the two-dimensional values of the reference hybridization patterns are clustered based on the supervised PLS-DA analysis.

The clustering is preferably done on the basis of the phenotypic characteristic for which the sample hybridization pattern is scrutinized. The clustering preferably results in two clusters, wherein one cluster has the certain phenotype and the other has not. The sample hybridization pattern can thus easily be identified as having or not having the certain phenotype. This typing is typically associated with a margin of error for the classification, i.e. the chance that the sample nucleic acid is wrongly classified as having or not having the certain phenotypic characteristic. The borders of the clusters can be set to accommodate a smaller or larger statistical chance of error. Thus a method of the invention preferably further comprises typing said sample nucleic acid on the basis of the presence or absence in a cluster. Preferably, a method of the invention, further comprising typing said sample nucleic acid on the basis of the presence or absence in a cluster. In a preferred embodiment the parameter comprises epidimicity. Preferably, the hybridization patterns are clustered or grouped on the basis of their epidemic phenotype, i.e. the potential for spread of the strain to other patients in a hospital setting.

The reference hybridization pattern can be generated from a wide variety of nucleic acids. As mentioned above, the reference hybridization pattern is preferably generated from a nucleic acid obtained or derived from a natural Staphylococcus aureus source. Preferably, at least 5 and more preferably at least 50 reference hybridization patterns are generated by nucleic acid obtained or derived from different Staphylococcus aureus strains. In a particularly preferred embodiment essentially all reference hybridization patterns are generated by nucleic acid of Staphylococcus aureus strains. In this way it is possible to type a sample for the presence therein of nucleic acid derived or comprises a similar phenotypic parameter as a certain strain or strains of Staphylococcus aureus. This particularly preferred embodiment is preferably combined with a statistical analysis of the reference and sample hybridization pattern. In this way it is possible to determine the chance that a Staphylococcus aureus in a sample comprises a certain phenotypic characteristic that is shared by some but not all Staphylococcus aureus strains. The Staphylococcus aureus nucleic acid can be derived from RNA but is preferably derived or obtained from Staphylococcus aureus DNA. i.e. derived from the genome. Thus in a preferred embodiment of the invention said plurality of nucleic acid molecules is derived from Staphylococcus aureus DNA.

An important advantage of a method of the present invention is now that it is not necessary that the strain is first taxonomically classified (e.g. identified) whereupon then the clinically relevant parameter(s) belonging to the identified strain can be determined, e.g. as based on a comparison with a list of data on known reference strains. Thus, it is an advantage of the present invention that no species-determination is required in order to determine the presence of, for instance, sensitivity (or resistance) of the test-organism to certain antibiotics, or any other clinically relevant parameter. This is achieved by the fact that such information is now provided “within” the plurality of nucleic acid molecules of the array.

The sample and/or reference nucleic acid used to generate the hybridization patterns may contain a subset of nucleic acid of the Staphylococcus aureus strain it is derived or obtained from. However, preferably no selections are performed. In any case, selections are preferably the same or similar for sample and reference nucleic acid. This allows for an easy comparison of the reference and the sample hybridization patterns.

With the term “nucleic acid obtained or derived from” it is meant that it is not essential that the nucleic acid used to hybridize on the array is directly obtained from the Staphylococcus aureus source. It may have undergone cloning, selections and other manipulations prior to the use for hybridizations. Sample and reference nucleic acid can for instance be obtained from cloned libraries, such as expression or genome libraries. Alternatively, sample and reference nucleic acid can be generated from scratch based on the nucleic acid information in databases, for instance, as a result of the ongoing genomics efforts. However, preferably sample and reference nucleic acid are obtained directly, or through amplification from a natural Staphylococcus aureus source. Preferably, the sample hybridization pattern is generated starting from a monoculture of a Staphylococcus aureus strain. In this way it is warranted that only one Staphylococcus aureus is analyzed on the array, and in the same time the hybridization pattern generated is a hybridization pattern generated from one Staphylococcus aureus strain.

In one aspect, the invention provides an array comprising a plurality of Staphylococcus aureus nucleic acid molecules wherein said nucleic acid molecules comprise an average size of between about 200 to 5000 nucleotides. An array of the invention preferably comprises at least 500.000 nucleotides on them. Preferably the arrays carry even more nucleotides on them. In a preferred embodiment the array comprises at least 1 megabase (10⁶ nucleotides). Preferably, they comprise at least 2 megabases. Contrary to conventional arrays, the number of bases per spot is high, i.e. between 200 and 5000 nucleotides. Preferably, said plurality of nucleic acid molecules are derived from a natural Staphylococcus aureus source. Preferably, wherein said plurality of nucleic acid molecules is derived from Staphylococcus aureus DNA. It has been found that different strains of Staphylococcus aureus, while belonging to the same species can nevertheless vary greatly in the amount and kind of DNA that they carry. Thus in a preferred embodiment, an array of the invention comprises a plurality of nucleic acid molecules that is derived from at least two different strains of Staphylococcus aureus. Preferably, the plurality of nucleic acid molecules in the array comprises at least a representation of the genome of a Staphylococcus aureus strain. This is preferably extended with nucleic acid derived from at least one other strain of Staphylococcus aureus. In this way the array is a more representative of the entire genetic diversity of the Staphylococcus aureus species. In a particularly preferred embodiment, the array comprises a plurality of nucleic acid molecules that is derived from at least three different strains of Staphylococcus aureus. By increasing the number of Staphylococcus aureus strains to generate the plurality of nucleic acids in the array, the array more and more mimics the complete genetic potential of the Staphylococcus aureus species and thus the typing becomes more and more specific. Thus in a preferred embodiment the array comprises a plurality of nucleic acid molecules comprising a representation of the genomic diversity of the Staphylococcus aureus species. This does not mean that typing with arrays carrying a reduced number of different Staphylococcus aureus strains is not a valid approach; it only means that predictions and estimations become more accurate and complete.

### EXAMPLE

### Clustering Based on a Distinction Between Epidemic and Non-Epidemic Staphylococcus aureus Strains by Supervised PLS-DA Analysis of Whole-Genome-Array Differential Hybridization Data

Fluorescently labeled genomic DNAs (gDNA) of a set of 31 different Staphylococcus aureus strains were separately hybridized to arrays coated with randomly chosen gDNA fragments of a mixture of 8 different S. aureus strains (approx. 2100 fragments/array, approx. 1500 bp/fragment). The fluorescent hybridization patterns were quantified resulting in a list of hybridizations signals per genomic DNA fragment for each tested strains. To be more specific each array was simultaneously hybridized with 2 labeled gDNAs: one concerning a specific S. aureus strains under investigation (labeled with Cy5), and the other concerning a standard mix of the 8 S. aureus strains used for making of the array serving as a reference to normalize hybridizations made on all the separate slides (labeled with Cy3).

Set of Different Bacterial Strains

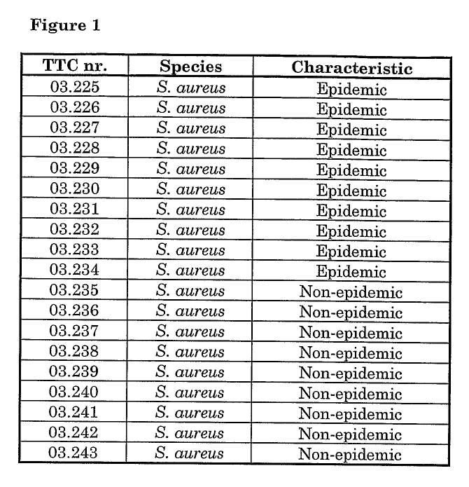

A set of 19 multiple resistant S. aureus strains was used for Example 3 (FIG. 5). The set consisted of 19 hospital isolates. For all strains their epidemic character was abstracted from daily hospital practice (FIG. 1). All experimental procedures used for the generation of micro-array results for these strains were according to the description in example 1.

Growth and gDNA Isolation of S. aureus Strains

S. aureus isolates were grown (via single colonies) on TSA-agar plates and/or TSA-medium (overnight, 37°) and stored as glycerol cultures (−80°). For gDNA isolation, plate grown bacteria (e.g. amount of 10-20 colonies) were resuspended in 400 μl TE-buffer (10 mM Tris-HCl, 1 mM EDTA, pH7.5) in a 2 ml vial. The cells were lysed by adding 400 μl water-washed 0.1 mm Zirconium glass-bead suspension (Biospec Products™), precooling on ice, medium-level shaking for 120 sec in a cell disrupter (minibeadbeater 8, Biospec Products™) and cooling on ice. After centrifugation (5 min, 14 krpm, 4° C.), gDNA was isolated from the cleared lysate according to standard procedures (Sambrook, 1989) by extraction with phenol/chloroform/isoamylalcohol (room temp.), extraction with chloroform/isoamylalcohol (room temp.), precipitation with ethanol/Na-acetate (−20° C., spinning at 4°), washing with 70% ethanol (−20° C., spinning at 4°), drying (vacuum), dissolving the pellet in 100 μl TE-buffer with RNAseA (1-100 μg/ml) and semi-quantification of the gDNA-amount on 0.6% agarose ethidiumbromide stained gels (e.g. 1-5 μl preparation/slot).

Construction of S. aureus gDNA Array (Slides)

To make an array containing a genome-wide representation of the species S. aureus, a mixed-genomic library of the organism was made by mixing gDNA of 8 S. aureus strains (for strain selection see FIG. 3). Strains were selected that: (a) showed each a different profile of resistance to a broad set of antibiotics (together covering most types of antibiotic-resistance), and (b) did not contain a significant plasmid band in the agarose-gel analysis of their isolated gDNA. The gDNA mix was sheared by sonication (Branson sonifier 450) and separated in several lanes of a 0.8% agarose gel. DNA-fragments (approx. 1-3 kb) were excised and isolated via binding to glass-milk (Bio101-kit). The isolated fragments were pretreatment with DNA-terminator End-repair kit (Lucigen Corp.) to facilitate efficient (blunt) cloning into bacterial plasmids (pSmartHCkan vector, CloneSmart Blunt Cloning Kit, Lucigen). Part of the ligation mix (1 μl) was transformed to 25 μl E. coli cells (E. kloni 10G supreme electrocompetent cells, Lucigen) by electroporation (0.1 cm-gap cuvets Eurogentec, BioRad Pulsor, 25 μF, 200 ohms, 1.6 kV) and regeneration in TB-culture medium and plating on TY-plates with 30 μg/ml kanamycin grown overnight at 37° C. Using tooth-picks, colonies were transferred to into 96-well microtiter plates (32 plates, 150 μl/well TY medium containing 30 μg/ml kanamycin). After overnight growing at 37° C., glycerol was added (final conc. 15%) and the glycerol-stocks were stored at −80° C.

The genomic inserts from each clone in the well-plates were multiplied using PCR-amplification in 96-well PCR-plates (22 plates). PCR reactions contained 50 μl reaction mix/well with 1× SuperTaq buffer, 0.2 mM of each dNTP (Roche Diagnostics), 0.4 μM primer L1(5′-cag tcc agt tac gct gga gtc-3′) and 0.4 μM primer R1(5′-ctt tct gct atg gag gtc agg tat g-3′), 1.5 U SuperTaq-DNA-polymerase and 1 μl glycerolstock from corresponding well of gDNA-bank. Both primers contain a free NH2-group coupled via a C6-linker to the 5′ end of the primer. The following PCR-program was used: 4 min 94° C., 30× (30 sec 94° C., 30 sec 50° C., 3 min 72° C.), 10 min 72° C. and soaking at 4° C. Following the amplifications, the 50 μl PCR-products were transferred to 96-well round-bottom plates and precipitated by adding 150 μl NaAc/isopropanol mix (0.2M NaAc, 67% isopropanol final conc. each), incubation 1 hr −80° C., spinning (1 hr, 2.5 krpm, 4° C.), removal of supernatant and washing with 100 μl 70% ethanol. DNA-pellets were resuspended in 50 μl water/well, transferred to 384-well plates, dried (speed vac) and resuspended in 10 μl 3×SSC-buffer per well. The 6 resulting 384-well plates, containing approx. 2100 PCR-products were used for spotting the micro-arrays. The PCR-products were spotted on series of maximal 75 “aldehyde” coated slides (Cell Associates)) using an ESI micro-array printer in combination with 24 TeleChem Stealth micro spotting quill-pins (approx 100 μm diameter). After spotting, slide surfaces were blocked by treatment at room temperature with boro-anhydride: 2×5 min in 0.2% SDS, 2×5 min in water, 10 min in boro-anhydride buffer (1.7 g NaBH4 in 510 ml PBS-buffer and 170 ml 100% ethanol), 3×5 min in 0.2% SDS, 3×5 min in water, 2 sec in 100° C. water, dry with N₂ flow. PBS (phosphate buffered saline) is 6.75 mM Na2HPO4, 1.5 mM K2HPO4, 140 mM NaCl, and 2.7 mM KCl pH 7.0. (1.2 g Na2HPO4, 0.2 g K2HPO4, 8.0 g NaCl, 0.2 g KCl per liter, pH 7.0).

Labeling of gDNA

Fluorescent labeling of gDNA was performed on 0.5-2 μg of isolated S. aureus gDNA for 1.5 hr at 37° in a 25 μl reaction based on BioPrimeR DNA Labeling System (Invitrogen, Cat. No.: 18094-011). The reaction contained (final conc): 1× RandomPrimer solution (50 mM Tris-HCl PH 6.8, 5 mM MgCl2, 30 μg/ml random octamers, Bioprime^{R}), 1× lowT dNTP-mixture (0.25 mM dATP, 0.25 mM dGTP, 0.25 mM dCTP, 0.1 mM dTTP), 0.06 mM Cy-dUTP (Cy=either Cy5 or Cy3, 1 μl of 1 mM stock, Amersham Biosciences) and 20 Units DNA-polymerase (Klenow fragment; 0.5 μl of 40 U/μl stock, Bioprime^{R}). After the reaction, salts, unincorporated (labeled) nucleotides and primers were removed by purification over an Autoseq G50 column (Amersham Biosciences). After purification 1/10^{th} part of the labeled material was used for spectrophotometric analysis to determine quantity of DNA (A^{260 nm}) and Cy5 (A^{649 nm}) or Cy5 (A^{550 nm}). The remainder of the labeled material was used for array hybridization.

(Pre-)Hybridization of Arrays

In preparation for hybridization, slides were laid in Petri dishes, in 20 ml prehybridization solution (1% BSA, 5×SSC, 0.1% SDS, filtered through a 0.45 μm filter, 42°) and were gently shaken (by mild rotation) for 45 min at 42°. Next the slide was washed 2× in 40 ml water (in a 40 ml capped tube) and quickly dried using an N₂-gun.

The appropriate gDNA samples that were labeled with Cy5-dUTP and Cy3-dUTP were combined with 4 μl yeast tRNA (25 μg/μl), dried (using a SpeedVac), re-dissolved in 40 μl EasyHyb solution (Roche Applied Science), denatured (1.5 min, 95° C.), spinned down briefly (1 sec, 10 krpm), pipetted on a pre-warmed (42° C. metal plate) dry prehybridized array, covered with a plastic cover slip (Hybrislip, Molecular Probes), inserted in a water-vapor-saturated preheated (42° C.) hybridization chamber (Corning) and hybridized overnight in a 42° C. water bath. For each hybridization gDNA from the tester strain was labeled with Cy5-dUTP, whereas a reference pool (mix of gDNAs from the strains which were used for array construction) was labeled with Cy3-dUTP. After hybridization the arrays were washed by shaking slides 4× in 40 ml of (different) buffers in capped 40 ml tubes (wash-buffer 1:1×SSC, 0.2% SDS, 37°, 5-10 sec; wash-buffer2: 0.5×SSC, 37° C., 5-10 sec; wash-buffer3 and 4: 0.2×SSC, 20° C., each 10 min).

Scanning and Image Analysis

After washing, slides were stored in the dark (to prevent decay of Cy-fluorescence) or directly used for scanning the fluorescent Cy dyes with a scanning device (ScanArray 4000 from PerkinElmer with Scanalyse software from Packard Bioscience). A quickscan (resolution 30 μm/pixel) was performed to select optimal laser-(intensity) and detection (photomultiplier) settings in order to prevent excess of low signals or saturated signals. Slides were 2 times scanned: for Cy5- and Cy3-fluorescence. Digital scans were quantified with ImaGene software (BioDiscovery Inc., version 4.2) resulting in a spot identity, and Signal (S) and Background (B) values for both Cy5 and Cy3, for each spot on the array. The data were stored in electronic files and used for further data processing.

Data Pre-Processing

By using spreadsheet software (Excel, Microsoft) the following calculations were made for each spot: S−B values for Cy3 and Cy5, Cy5/Cy3 ratio's [R=Cy5(S−B)]/[(Cy3(S−B)]. Low quality data were removed (e.g. spots having Cy3 data with S<2B). Then, a normalization factor N was calculated for each slide, based on average Cy5- and Cy3-signal for all spots on a slide (N=[averageCy5(S−B)]/[averageCy3(S−B)]. Next, normalized ratio's (Rₙ) were calculated for each spot (Rₙ=R/N) on all arrays. A matrix (=dataset) of normalized ratio's per spot for many slides (slides relate with S. aureus strains) was used for further data preprocessing.

Since the Cy3 signal is generally present for most spots (Cy3-labeled reference gDNA pool of 8 strains was hybridized to all slides), and the Cy5 signal can vary (Cy5-labeled gDNA of different strains were each hybridized to single slides), the Cy5/Cy3 ratio can in theory have two values (1 or 0) if a gDNA fragment is present or absent respectively, in the Cy5 tested strain. In practice, however, these values vary around 1 and 0. Therefore, in many analyses cut-off values for 0 and 1 were applied on the ratio-dataset before further analysis (e.g. Rₙ<0.5 and Rₙ>0.5 were replaced by 0 and 1 respectively, or, Rₙ<0.3 and Rₙ>0.7 were replaced by 0 and 1 respectively while keeping Rₙ values between 0.3 and 0.5). These “cut-off datasets” were used for the final data analysis.

Results

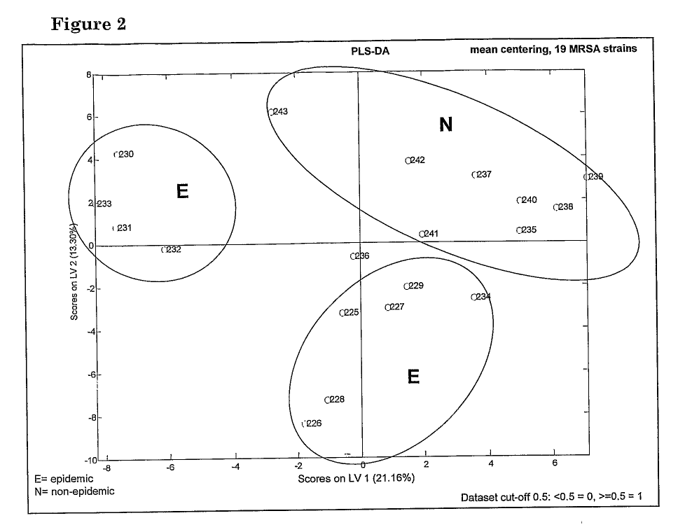

A set of 19 different S. aureus isolates was processed for PLS-DA analysis of data from differential hybridization on whole-genome micro-arrays (FIG. 2).

The PLS-DA analyses resulted in significant clustering of the S. aureus strains according to their known epidemic character (FIG. 2, E=Epidemic, N=Non-epidemic).

This shows that part of the total differential hybridization dataset contains predictive information that can be used to predict epidemicity of unknown S. aureus strains

1. A method for typing sample nucleic acid derived or obtained from a Staphylococcus aureus strain, comprising:
providing an array comprising a plurality of nucleic acid molecules, wherein said plurality of nucleic acid molecules is derived from a first set of at least two different strains of Staphylococcus aureus;
providing at least two different reference hybridization patterns by hybridizing said array with at least two different reference nucleic acids obtained or derived from a second set of at least two different strains of Staphylococcus aureus, wherein said strains of Staphylococcus aureus in said second set are separable into at least two groups on the basis of a value for at least one phenotypic parameter;
creating at least two different clusters of reference hybridization patterns by clustering the reference hybridization patterns by unsupervised multivariate analysis;
hybridizing the same array as used for preparing the reference hybridization patterns with sample nucleic acid to obtain a sample hybridization pattern, and
assigning the sample hybridization pattern to one of said at least two different clusters of reference hybridization patterns.

2. A method according to claim 1, wherein the average size of the fragments in said sample nucleic acid is between about 50 to 5000 nucleotides.

3. A method according to claim 1, wherein the average size of the molecules in said plurality of nucleic acid molecules is between about 200 to 5000 nucleotides.

4. A method according to claim 1, wherein said array comprises between about 1.500 and 5.000 nucleic acid molecules randomly chosen from fragments of the fragmented genomic DNA of said at least two different strains of Staphylococcus aureus.

5. A method according to claim 1, wherein said sample nucleic acid is derived from a pure culture of Staphylococcus aureus.

6. A method according to claim 1, wherein said plurality of nucleic acid molecules is derived from at least three, preferably at least 5, and even more preferably at least 8 different strains of Staphylococcus aureus.

7. A method according to claim 1, wherein said method comprises comparing the sample hybridization pattern with clusters of reference hybridization patterns comprising at least 3, more preferably at least 5 and even more preferably at least 50 different reference hybridization patterns.

8. A method according to claim 1, wherein said phenotypic parameter is the epidemicity of said strains.

9. A method according to claim 7, wherein said comparison comprises Partial Least Square-Discriminant Analysis (PLS-DA) of the reference hybridization patterns together with the sample hybridization pattern and wherein at least one phenotypic parameter of which the values are known for the reference hybridization patterns, (and which information is used to supervise the PLS-DA analysis), is additionally determined or estimated for the sample nucleic acid or the source it is derived from.

10. A method according to claim 9, further comprising clustering hybridization patterns based on the supervised PLS-DA analysis.

11. A method according to claim 1, wherein essentially all reference hybridization patterns are generated by nucleic acid of Staphylococcus aureus strains.

12. A method according to claim 1, wherein said typing comprises determining whether the Staphylococcus aureus in said sample nucleic acid is derived from is an epidemic strain.

13. A method according to claim 1, wherein said second set of at least two different strains of Staphylococcus aureus comprises strains from said first set.

14. A kit of parts, said kit comprising a combination of an array as defined in claim 1 together with at least two different ones of said reference hybridization patterns or said reference nucleic acids.

15. A method according to claim 2, wherein:
the average size of the molecules in said plurality of nucleic acid molecules is between about 200 to 5000 nucleotides;
said array comprises between about 1.500 and 5.000 nucleic acid molecules randomly chosen from fragments of the fragmented genomic DNA of said at least two different strains of Staphylococcus aureus;
said sample nucleic acid is derived from a pure culture of Staphylococcus aureus;
said plurality of nucleic acid molecules is derived from at least three, preferably at least 5, and even more preferably at least 8 different strains of Staphylococcus aureus;
said method comprises comparing the sample hybridization pattern with clusters of reference hybridization patterns comprising at least 3, more preferably at least 5 and even more preferably at least 50 different reference hybridization patterns;
said phenotypic parameter is the epidemicity of said strains;
said comparison comprises Partial Least Square-Discriminant Analysis (PLS-DA) of the reference hybridization patterns together with the sample hybridization pattern and wherein at least one phenotypic parameter of which the values are known for the reference hybridization patterns, (and which information is used to supervise the PLS-DA analysis), is additionally determined or estimated for the sample nucleic acid or the source it is derived from;
said method further comprises clustering hybridization patterns based on the supervised PLS-DA analysis;
essentially all reference hybridization patterns are generated by nucleic acid of Staphylococcus aureus strains;
said typing comprises determining whether the Staphylococcus aureus in said sample nucleic acid is derived from is an epidemic strain;
said second set of at least two different strains of Staphylococcus aureus comprises strains from said first set.

16. A kit of parts, said kit comprising a combination of an array as defined in claim 15 together with at least two different ones of said reference hybridization patterns or said reference nucleic acids.
